(19) United States
(12) Patent Application Publication
(10) Pub. No.: US 2016/0356704 A1
(43) Pub. Date: Dec. 8, 2016

(54) NONDESTRUCTIVE MEAT TENDERNESS ASSESSMENT

(71) Applicant: PURDUE RESEARCH FOUNDATION, West Lafayette, IN (US)

(72) Inventors: Young L. Kim, West Lafayette, IN (US); Yuan Hwan Brad Kim, West Lafayette, IN (US); Taehoon Kim, West Lafayette, IN (US)

(73) Assignee: PURDUE RESEARCH FOUNDATION, West Lafayette, IN (US)

(21) Appl. No.: 15/175,259

(22) Filed: Jun. 7, 2016

Related U.S. Application Data
(60) Provisional application No. 62/172,149, filed on Jun. 7, 2015.

(51) Int. Cl. G01N 21/359; G01N 21/3563; G01N 33/12; G01J 3/28; H04N 5/232; H04N 5/225

(52) U.S. Cl. CPC G01N 21/359; H04N 5/232; H04N 5/2256; G01N 33/12; G01J 3/2823; G01N 21/3563; G01N 2201/068; G01N 2201/062; G01N 2201/061; G01N 2201/0638

(57) ABSTRACT

A novel non-invasive anisotropic image scanning technology assessing tissue structural changes is disclosed for use as a non-invasive and rapid meat quality prediction tool.

### CROSS-REFERENCE TO RELATED APPLICATIONS

The present U.S. patent application is related to and claims the priority benefit of U.S. Provisional Patent Application Ser. No. 62/172,149, filed Jun. 7, 2015, the contents of which is hereby incorporated by reference in its entirety into this disclosure.

### TECHNICAL FIELD

The present disclosure generally relates to meat assessment techniques, and in particular to a method for assessing meat tenderness nondestructively using tissue anisotropy imaging.

### BACKGROUND

This section introduces aspects that may help facilitate a better understanding of the disclosure. Accordingly, these statements are to be read in this light and are not to be understood as admissions about what is or is not prior art.

Numerous factors influence the development of meat quality, such as the individual animal (breed, sex, age), environmental conditions (including feeding, transporting and slaughtering conditions), and processing (e.g., storing time/temperature condition) (Liu et al., 2002). Changes in these factors can affect the development process of the meat and, in the end, the final product itself. Maintaining these factors to be constant at every given time can prove to be extremely difficult and attempts to control them have been proven to be a difficult challenge for the meat and food industries. These variation changes can be reflected as a variation in color, tenderness, flavor and overall quality of the meat, which in turn will adversely affect consumer satisfaction. Both tenderness and color are primary factors closely related to meat quality and consumer satisfaction. Meat tenderness is an attribute that has always been asked by consumers when they are purchasing meat, while meat color is used as the main deciding factor for a consumer purchasing decision.

The Warner-Bratzler shear force (WBSF) is a widely used instrumental measure of meat tenderness (Wheeler & Koohmaraie, 1997) and Hunter LabScan for the measure of meat surface color (Yacowits, Davies & Jones, 1978). While the WBSF is generally reliable and accurate, it is an undeniably destructive method that requires a lot of time and labor during the process. These tasks increase the cost of the product since in order to provide meat with constant quality, these methods have to be performed repeatedly by the industries. There is therefore an unmet need for a novel, non-destructive, rapid, and straightforward method to make the quality testing less demanding.

### SUMMARY

In one aspect, a hyperspectral imaging system is disclosed, which includes a detector and a broadband light source.

In another aspect, a method for assessing meat tenderness is disclosed which includes illuminating a tissue sample with a light, varying at least one parameter of the light with a spectrometer, and forming an image of the tissue sample by collecting backscattered light from the tissue using a lens system.

In yet another aspect, a method for assessing meat tenderness is disclosed, which includes illuminating broadband light from a light source onto a sample, resolving wavelength information using a filter, and obtaining an image set of different wavelengths of the sample using a detector.

### BRIEF DESCRIPTION OF THE FIGURES

FIG. 1 is a picture of an embodiment of the herein disclosed hyperspectral imaging system.

FIG. 2 is a graph showing the Warner-Bratzler shear force (WBSF) measurement results over different aging periods for LD at 1 d display.

FIG. 3 is a graph showing tissue anisotropy-weighted reflectance measurement results over different aging periods for LD at 1 d display.

FIG. 4 is a plot of a comparison of averaged WBSF and tissue anisotropy-weighted reflectance over different aging periods for LD at 1 d display.

FIG. 5 is an image of a steak sample captured using a DSLR camera.

FIG. 6 is an image of the steak sample of FIG. 5 captured using the herein described anisotropic imaging system.

FIG. 7 shows WBSF values of loins over different postmortem aging periods.

FIG. 8 shows reflectance values of loins over different postmortem aging periods.

FIG. 9 shows images of representative scattering anisotropy imaging (SAI) results, compared with conventional digital photography.

FIG. 10 is a photograph of an embodiment of the herein described SAI system; telecentric imaging with a coaxial illuminator for light illumination.

FIG. 11 is a diagram demonstrating a detection mechanism of SAI for meat tenderness assessment.

FIG. 12 is an image of an embodiment of the herein disclosed system showing integration of a telecentric lense and a smartphone.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In response to the unmet need, presented herein is a novel, non-destructive, rapid and straightforward method to allow for assessment of meat tenderness.

An anisotropic imaging system is disclosed herein for analyzing biological tissues. The scattered angle at the tissue surface after the light propagation is varied by how the light travels inside, which is determined by the tissue internal structures. Thus, under back-directional (filtering) imaging, the light intensity backscattered from biological tissue is mainly sensitive to the scattering anisotropy factor, which is highly associated to subtle alterations in tissue structures and organizations. This imaging method is distinctly different from biomedical-focused imaging methods, which rely on pattern-recognition algorithms of photographed images. In particular, it has been shown to effectively detect the changes in skin tissue due to the development of cancer cells.

In a mesoscopic imaging system, a back-directional (angular) filter collects a small solid angle to form an image in the exact backward direction (Xu et al., 2012). Back-directional angular gating provides an image with a high tissue microstructure contrast due to the highly anisotropic nature and the angular scattering distribution of tissue is sensitive to tissue architecture in the backward direction. The system also enhanced the field of view, image resolution, contrast and the depth of the image captured. In this generation, Xu et al. moved forward on the notion that highly collimated (or directional) light illumination was strictly necessary for mesoscopic tissue imaging, in addition to back-directional angular filtering detection. Conventionally, it is expected that the illumination directionality is required to match with the detection directionality for back-directional angular filtering. In the second generation of the system, a telecentric lens system is adopted. The lens can provide image with a more enhanced field of view, resolution, and depth. With applying different illumination wavelength, the field of view, depth and resolution of the image are enhanced further. The telecentric lens has been commonly used in the machine vision industry for a packaging inspection and verification, but rarely used in a grading system.

Importantly, as disclosed herein, it is not necessary to incorporate highly collimated illumination for effective mesoscopic tissue imaging so long as a means of back-directional angular filtering (or gating) is used during detection, i.e., image formation. This finding allows any type of illumination (either highly collimated illumination or diffusive illumination onto tissue) to be used to achieve effective imaging of biological tissue on a mesoscopic scale. While most biological tissue has a high anisotropy factor (ranging between about 0.8-0.9), which is defined by the average cosine of the scattering angle with respect to the incident light, the anisotropy factor of tissue is not 1.0. Thus, it is not necessary to have highly collimated (or directional) light illumination, and the herein described system is therefore well tolerating of diffuse light illumination. That is, for illumination geometries, there can be two different schemes collimated illumination or diffuse illumination (from a ring illuminator). In the case of the herein described system, both can be used.

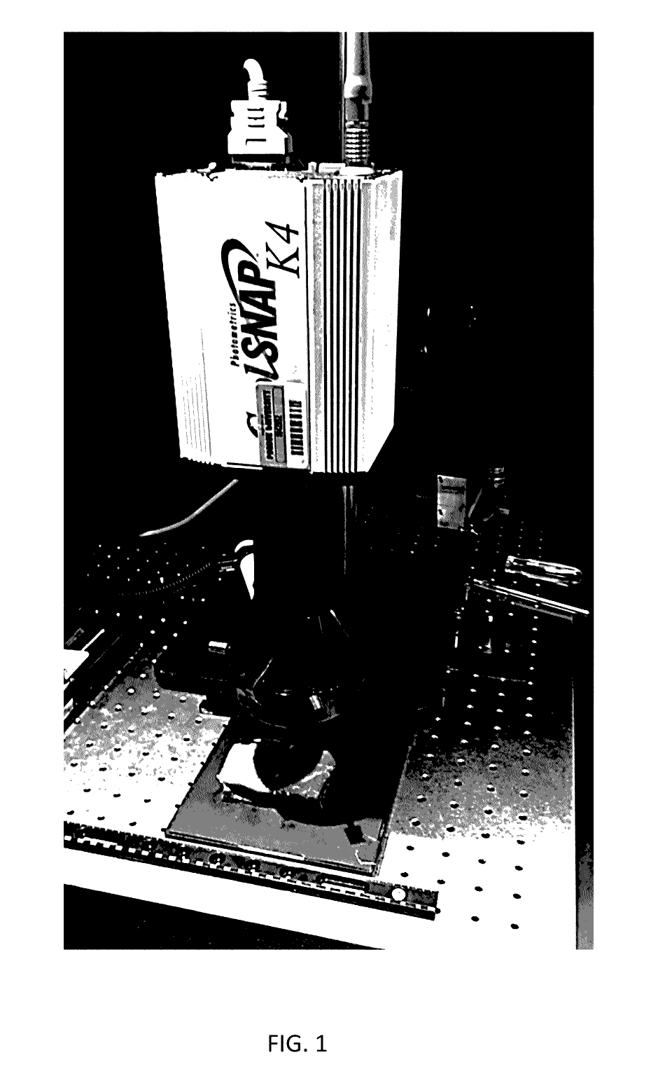

Referring to FIG. 1, in one aspect, a hyperspectral imaging system is disclosed, which includes a detector and a light source. In an embodiment, the detector can be a camera. The camera can be a charge-coupled device (CCD) camera. In another embodiment, the camera is a complementary metal oxide semiconductor (CMOS) camera. In yet another embodiment, the light source includes a white-light source. The white-light source can include any one of or a combination of light emitting diodes, xenon, and tungsten lamps.

In another aspect, method for assessing meat tenderness is disclosed. The method can include the steps of illuminating a tissue sample with a light, varying at least one parameter (which can be the wavelength) of the light with a spectrometer, and forming an image of the tissue sample. The image of the tissue sample can be formed by collecting backscattered light from the tissue using any one of or a combination of a small aperture 4-focal length (4-f) lens system within an angular cone of 2°-5°, a telecentric lens, and an anti-scatter grid and a detector lens. The light can be diffused light and/or collimated light. In an embodiment, the method of claim can further include coupling and decoupling scattering and absorption contributions in the tissue sample.

In yet another aspect, a method for assessing meat tenderness is disclosed. The method can include the steps of illuminating broadband light from a light source onto a sample (which can be a tissue sample), resolving wavelength information using a filter, and obtaining an image set of different wavelengths of the sample using a detector. In an embodiment, the broadband light can be any one of or a combination that includes visible light and near infrared light. The visible and near-infrared light wavelength range can be about 400 to about 1400 nm. In another embodiment, the filter can include any one of or a combination of a mechanical color filter, a liquid crystal tunable filter, and an imaging spectrograph.

As demonstrative of the principles disclosed herein, a study has been performed, the results of which are herein reported, to observe the applicability of the herein disclosed novel anisotropic imaging system as a non-invasive instrument to assess both meat tenderness and color over various postmortem aging and under retail display conditions. Tenderness and color measured using standard method (WBSF and Hunter) are also conducted as a standard comparison.

Materials and Methods: /

1. Sample Preparation:

A total of 3 steers were slaughtered (±16 months old). At 7 days (d) post mortem, both M. longissimus dorsi (LD) and M. semitendinosus (ST) from one side if the carcass were removed. Each muscle was then divided into 4 parts, randomly allocated to 4 different aging times (7, 14, 21, and 28 d) and weighed. All samples excluding 7 d were individually vacuum-packaged and aged at 2° C. The pH of each sample was measured in triplicate using calibrated Hanna HI 99163 Meat pH meter. Three steaks, one steak for Warner-Bratzler Shear Force (WBSF) measurement (2.4 cm thick) and two steaks for display (1 d and 4/7 d; 2.4 cm thick; 10 cm×8 cm) were collected. Display steaks were packed using an overwrap-PVC film, and displayed for 7 d at 2° C. under continuous fluorescent natural white light (1600 l×). WBSF steak was immediately cooked, stored at 2° C. for 24 h prior to the measurement. At 4 d of display, 1 d display sample was taken of display and used for WBSF measurement. One sample for bio-chemistry testing was collected from each sample group, individually vacuum-packaged and stored at −80° C.

2. Color Measurement:

Color was measured and evaluated at 1, 4, and 7 d of displaying periods. Surface colors of the steaks were measured through the overwrap-PVC film in triplicates using calibrated HunterLab MiniScan EZ 4500L. CIE L*a*b* values were used to calculate hue angle (for discoloration, [b*/a*]^{tan-1}) (AMSA, 1991). Sensory color evaluation for both surface lean color and discoloration was evaluated by trained panelist (n=8).

Cooking Protocol:

To cook the sample, cooking griddle was first set to heating temperature of 290° F. (143° C.) to heat the griddle. After it reached 290° F. (143° C.), the griddle temperature was then adjusted to cooking temperature of 275° F. (135° C.). Initial and final cooking weight were measured after removing excess water to obtain the cooking loss. Prior placing the steak on the grill, a thermocouple (TruTemp 3519N) was inserted into the center of the steak to monitor the internal temperature of the steak. When the internal temperature of the steak reached 106° F. (41° C.), the steak was turned over to cook the other side. The cooking process continued until the internal temperature reached 160° F. (71° C.). The steak was then wrapped using aluminum foil and cooled at 2° C. for 24 h.

Warner-Bratzler Shear Force Measurement:

After 24 h of cooling at 2° C., six cores parallels to the fiber direction were collected from each sample. Sample was sheared using calibrated TA-XT Exponent Stable Micro System adjusted for WBSF measurement. Sample was sheared perpendicular to fiber direction. The average peak shear force of the six cores was calculated and used for the analysis.

Anisotropic Image Scanning:

Sample was scanned using Anisotropic Imaging System in a Purdue University's Weldon School of Biomedical Engineering Research Laboratory. White-light illuminator is selected using liquid crystal tunable filter and is illuminated onto sample via rig illumination. Light reflected is collected by a telecentric lens and is recorded using a CCD camera. The wavelength is varied from 400 nm to 720 nm with spectral resolution of 10 nm. A total of three images scan was collected from each sample. The reflectance intensity images were analyzed to avoid the strong absorption from myoglobin.

Results:

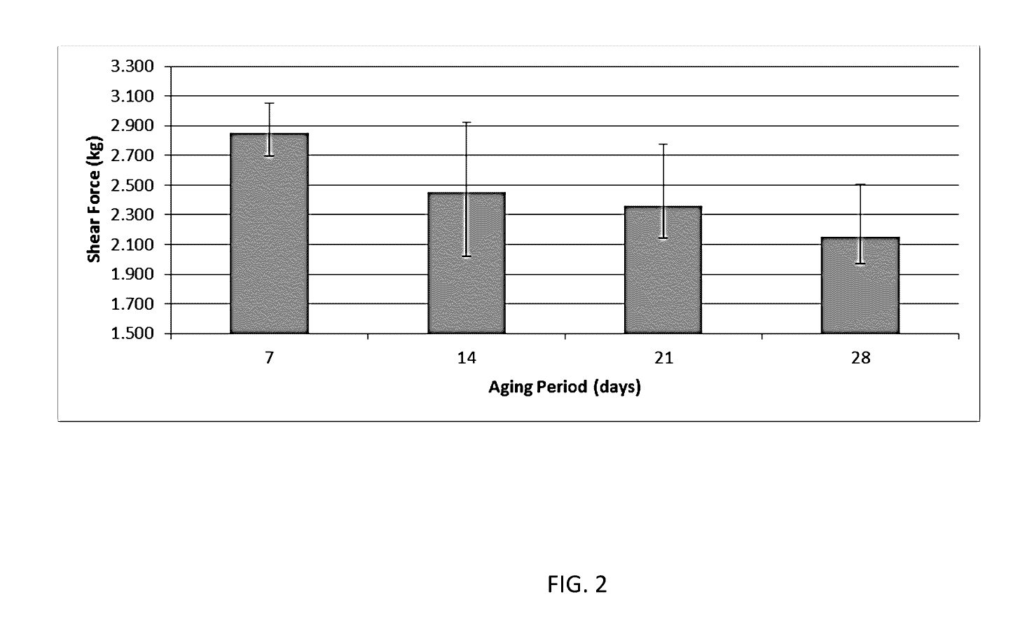

Referring to FIG. 2, the data obtained from both WBSF measurement and reflectance from three animals was averaged for the analysis process. Both methods showed a decreasing trend over the different aging periods.

As seen in FIG. 2, the measurement obtained from WBSF was significantly decreasing over a longer aging period (P<0.05). The smaller force required to cut through the meat indicated the increase of tenderness in the meat. This result was in accordance to the expectation which was the longer aging time would increase the tenderness of the meat as more muscle proteins were degraded during the process.

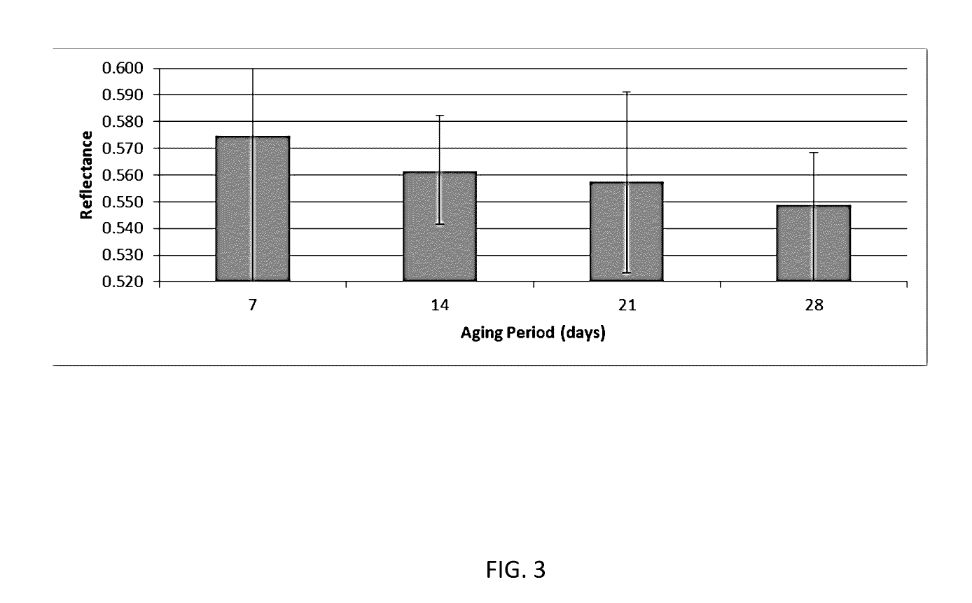

FIG. 3 shows that the reflected light detected by the system significantly decreased as the aging period was increased (P<0.05). This showed that the muscle structure changes due to the muscles protein degradation could be detected by the imaging system.

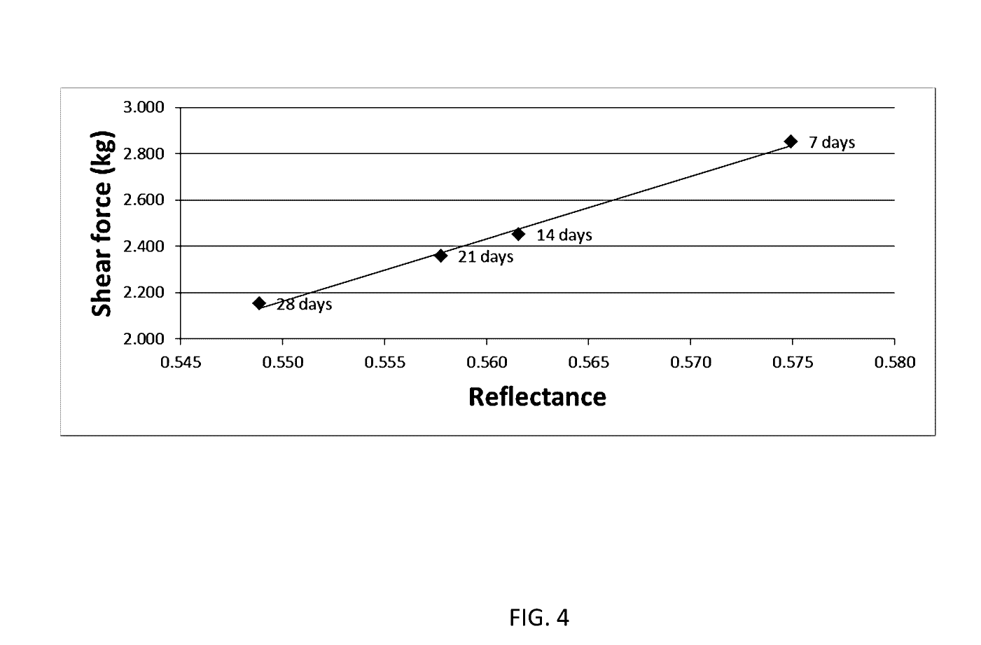

FIG. 4 indicates that both measurement methods showed an obvious similar linear trend over different aging time. The longer the aging period, the smaller the WBSF and reflectance value obtained. The R² of the line was 0.995 (P=0.003). These values suggested a very strong correlation between the two methods in assessing meat tenderness.

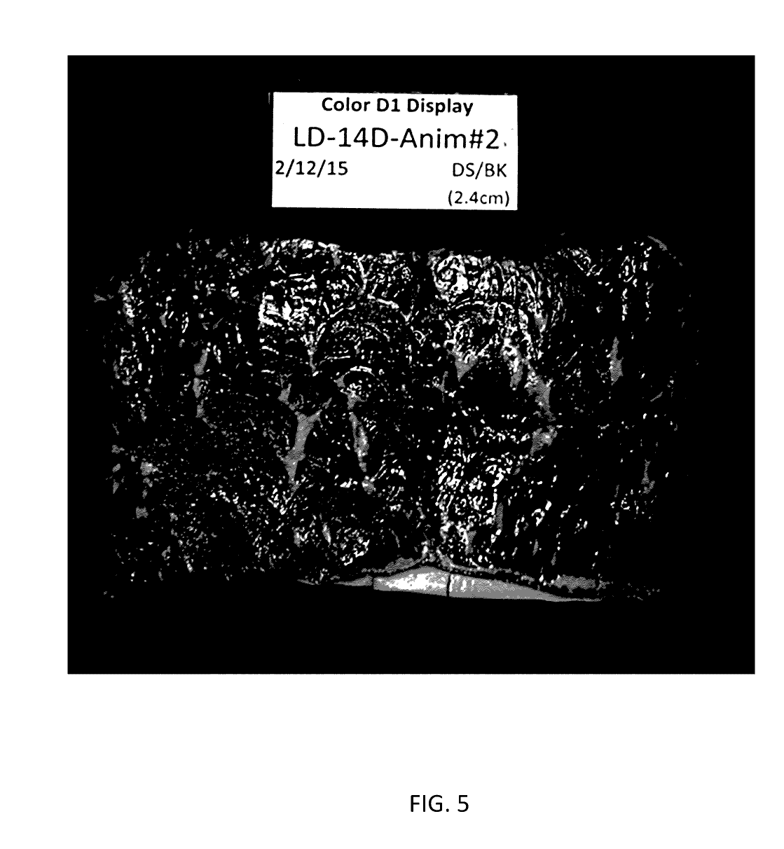

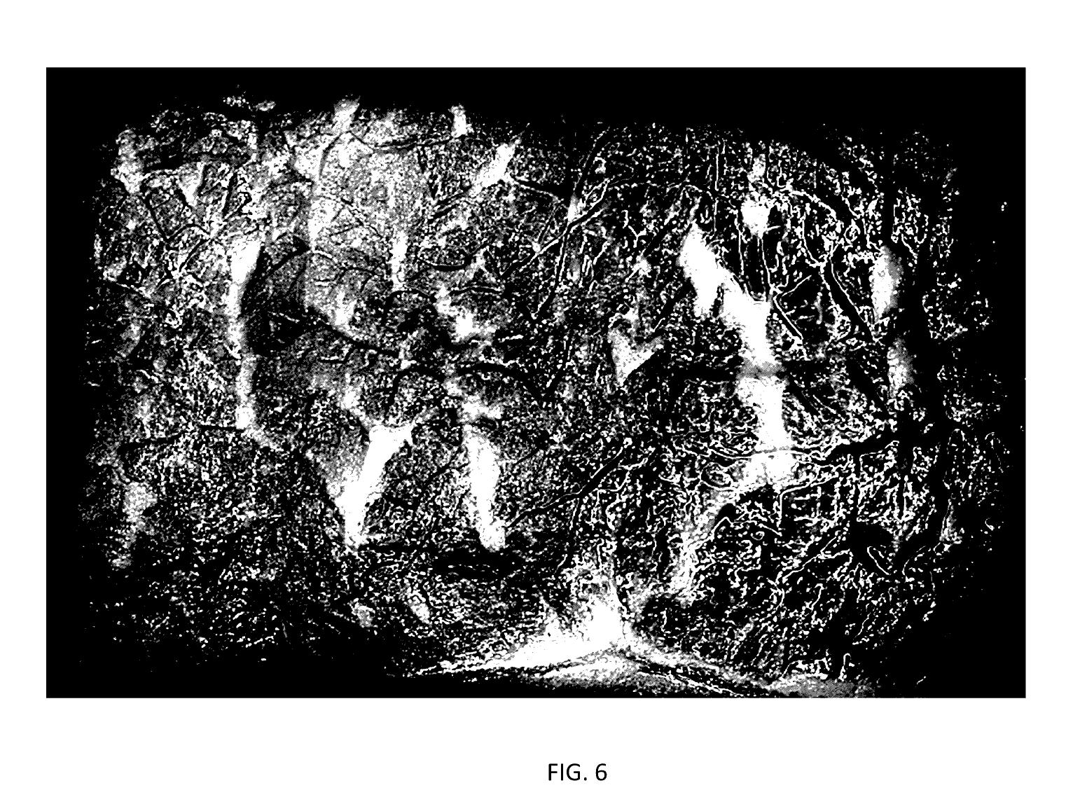

FIG. 5 shows an image of a steak sample captured using a DSLR camera. FIG. 6 shows an image of the steak sample of FIG. 5 captured using the herein described anisotropic imaging system.

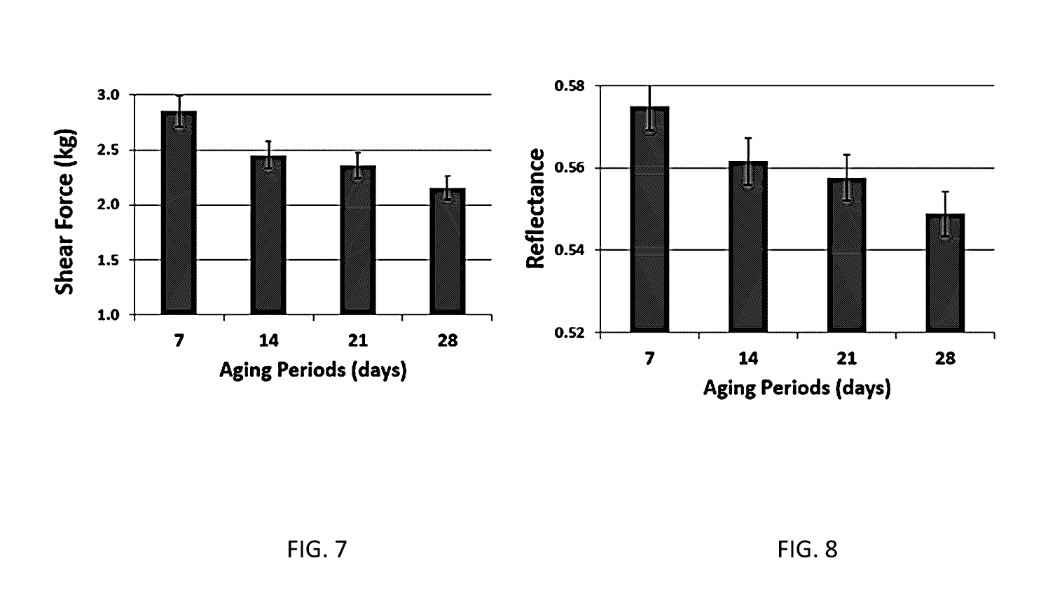

In another study, feasibility of the non-invasive SAI analysis for assessing the tenderness of beef strip loins (M. longissimus lumborum) at various postmortem aging periods (7, 14, 21 and 28 d). At each aging time, two steak cuts (2.4-cm thick; 10×8 cm²) were made for the Warner-Bratlzer shear force (WBSF) measurement and the SAI analysis, respectively. For the image analysis, the reflectance intensity of images, which were mainly determined by the scattering anisotropy factor, was averaged over the entire area. Significant decreases were found in both WBSF and SAI reflectance values of loins with aging periods (P<0.05), indicating gradual enhancement in meat tenderness with aging as expected (FIG. 7). Furthermore, we found an excellent linear correlation of both averaged SAI reflectance and WBSF values with a R² of 0.995 (P=0.003) with aging (FIG. 8). For the data in FIGS. 7 and 8, the image of the beef samples captured using the anisotropy image analysis system and averaged cosine values of the scattering angle were calculated (Kim et al., 2016). These results indicate that meat tenderness can be quantified in a non-destructive, consistent, and highly accurate manner by the herein described novel imaging system.

Discussion:

As mentioned above, novel, non-invasive and rapid detection method for analyzing meat quality has been one of the major interest in the last decade for the meat industry as the traditional methods for assessing meat quality are time consuming, destructive and inconsistent (ElMasry et al., 2011). Various detection methods including several imaging systems have been introduced as potential solutions to this problem. The most common and studied imaging methods are the hyperspectral imaging system and the near-infrared imaging system. All these imaging systems have a similar methodology in which lights are used in order to obtain the information from the sample. The system is also commonly coupled with a computer vision technology making it possible for an automated measurement and analysis, which greatly improve the consistency and accuracy of the data. The main differentiating factors of these imaging systems are the light source type it utilizes and the method used in capturing the reflectance.

In the hyperspectral imaging system, a light source with a visible wavelength is commonly used. This system is commonly used to highlight the surface of an object. It uses the principal of a spectrophotometer, in which only a single wavelength of the visible light spectrum is used in a given period of time of the scanning. This method provides both spatial and spectral images of the sample, increasing the amount and depth of the information that can be gathered from it. The wavelengths of the light transmitted are also able to penetrate the sample and causing scattering in the reflectance, which contains a substantial information on the internal structure of the product (Jackman et al., 2011). The near-infrared imaging system also applied the same measuring principle as the hyperspectral imaging system. This system is normally coupled with the hyperspectral imaging system; however, instead of visible light spectrum wavelength, the system utilizes 900-1700 nm near-infrared wavelength during the scanning process. The novel anisotropic imaging system also applies the hyperspectral imaging system scanning method in its system. A single wavelength of the visible light spectrum is transmitted to the sample in a given time of the scanning process.

The main difference of the systems appear after the light being transmitted onto the sample. In both hyperspectral imaging system and near-infrared imaging system, the diffused reflectance commonly collected with backscattering angle of around 30-35°, providing image with <1 mm depth. In the novel anisotropic imaging system presented herein, a back-directional gating collection system are applied in the anisotropic imaging system. The back-gating capturing system allows us to be able to reduce the collections angle to 2°, hence effectively removing the diffused light reflected. The back-directional gating system also capturess the reflectance in a backward direction (reflection) since meat structure is more sensitive in the backward direction due to its anisotropic properties. Anisotropic means that it is directionally dependent. In the case of meat products, this means that the reflected light comes out in a different direction depending on the structure of the meat and hence reducing the amount of available reflectance to be captured. The anisotropic imaging system is able to minimize this random direction reflectance factor, and thus able to create an image with a unique image contrast. The system also utilizes a telecentric lens, which increases the sensitivity, field of view, resolution and imaging depth of the imaging system, thus able to provide an image with a higher unique contrast of the tissue microstructure and a depth of 1.2 mm compared to the previous imaging system.

Such modifications in the systems causes each system to generate a different result. In the near-infrared imaging system, 900-1700 nm near-infrared wavelength during the scanning process. Due to the usage and nature of infrared light, other than the physical properties of the sample, complete information on the chemical constituent in a sample can also be gathered (ElMasry et al., 2012). Based on a study by Rust et al. (2008), a low correlation coefficient between the observed shear force and predicted shear force value was founded, indicating that the system is not accurate in predicting tenderness. From the same study, the near-infrared imaging system only able to go up until 70% tenderness certification level, which means only 70% of the scanned product is actually fully meet the tenderness level. When the hyperspectral imaging system and near-infrared system were coupled, the prediction level increased. In the previous study, it was observed that tenderness measured using this system is viable but not highly accurate. From the study conducted by ElMasry et al. (2012), the predicted shear force obtained using the imaging system when compared to the measured shear force produced a strong correlation with an R² value of 0.83. Though having strong correlation, another study by Cluff et al. (2013), showed that the accuracy of the system does not changed much, showing the system only able to go as far as 75% tenderness certification level. Large latent factors as well as high values of error in calibration and cross validation indicates that the model is not robust (ElMasry et al., 2012). The novel anisotropic imaging system presented herein, based on the above summarized study, showed a stronger correlation and accuracy in predicting meat tenderness. The measured WBSF and reflectance from the imaging system was correlated and an R² of 0.995 (P=0.003) was calculated. Data show a strong correlation between the two methods and indicates that the system can captured the microstructure of the sample and therefore create a more accurate prediction.

The main goal of the above summarized study was to analyze the viability of the herein disclosed novel anisotropic imaging system as a non-invasive and rapid beef quality prediction tool. Two main effects observed in this study were the muscle and aging effect. LD and ST muscle were chosen to observe the muscle sample due to the fact that ST has a higher amount of connective tissue, which will affect the overall tenderness. Four different aging times (7, 14, 21, and 28 d) were used in order to see the aging effect.

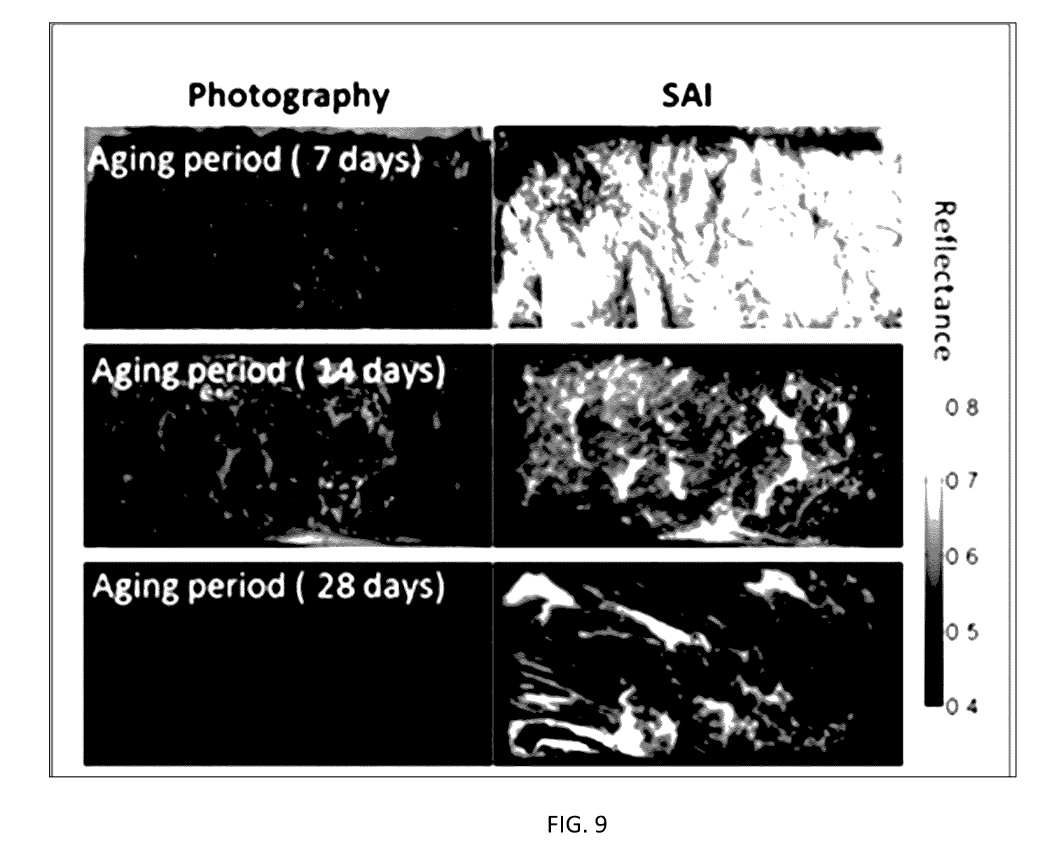

From this study, a significant decrease in WBSF measurement (P<0.05) was observed, indicating an increase in meat tenderness with aging. The reflectance from the anisotropy image taken also significantly decreased as aging time increased (P<0.05). The tenderness measured was correlated with the anisotropy images taken and showed a really strong correlation with an R² of 0.995 (P=0.003). The results indicate that the novel tissue anisotropic imaging system disclosed herein can be useful as a non-invasive and rapid tool in determining the extent of meat tenderization. FIG. 9 shows images of representative scattering anisotropy imaging (SAI) results, compared with conventional digital photography. The reflectance intensity, which is normalized by a reflectance reference standard, is inversely correlated with the aging period (Kim et al. 2016).

### EXAMPLE

### Sensitively, Reliably, and Non-Destructively Quantifying Beef Tenderness by Reflectance Intensity Images Obtained from the herein Described Anisotropic Imaging Technology

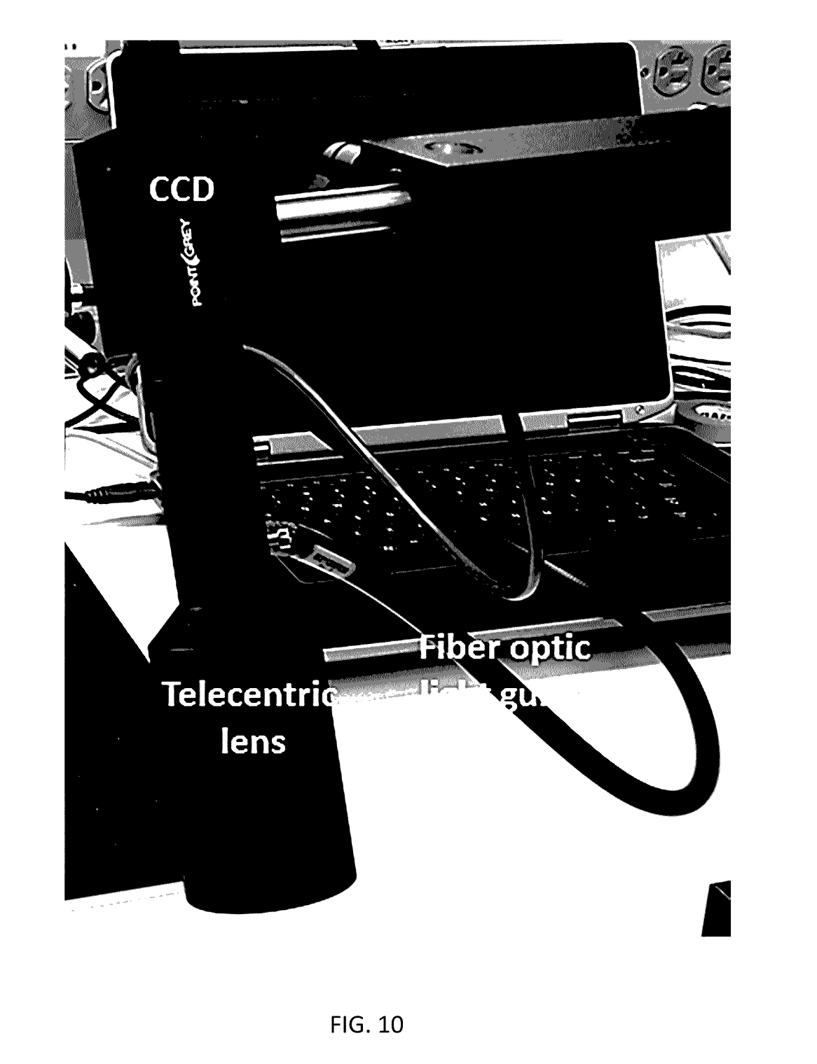

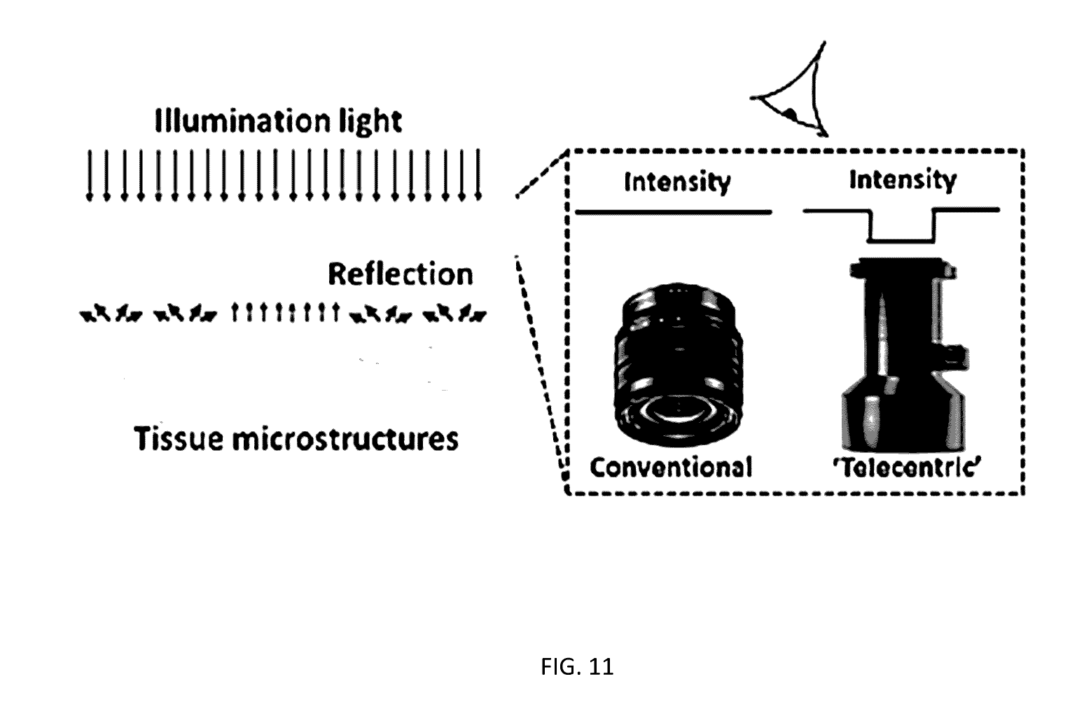

Referring to FIGS. 10 and 11, an example embodiment takes advantage of telecentric lenses and implements a novel imaging configuration in a reflectance imaging system. Under telecentric imaging, the light intensity backscattered from biological tissue is mainly determined by the scattering anisotropy factor, which is highly sensitive to subtle alterations in tissue structures and organizations, in particular the extracellular matrix (e.g. collagen matrix and cytoskeleton myofibrillar protein remodeling and realignment).

Referring to FIG. 11, the scattered angles at the meat surface are varied by how the light travels inside, which is determined by the internal tissue structures. Thus, ‘telecentric’ imaging is distinct from conventional imaging, allowing for direct assessment of meat tenderness. Still referring to FIG. 11, the exit angle of the light backscattered from meat at the surface can vary depending on how the light travels inside, which, in turn, is determined by the tissue internal structures. Therefore, under ‘telecentric’ imaging, the light intensity backscattered from meat can sensitively capture the scattering anisotropy factor (Konger et al. 2013; Visbal-Onufrak et al. 2016; Xu et al. 2012), which, in turn, can be used to predict the degree of meat tenderness.

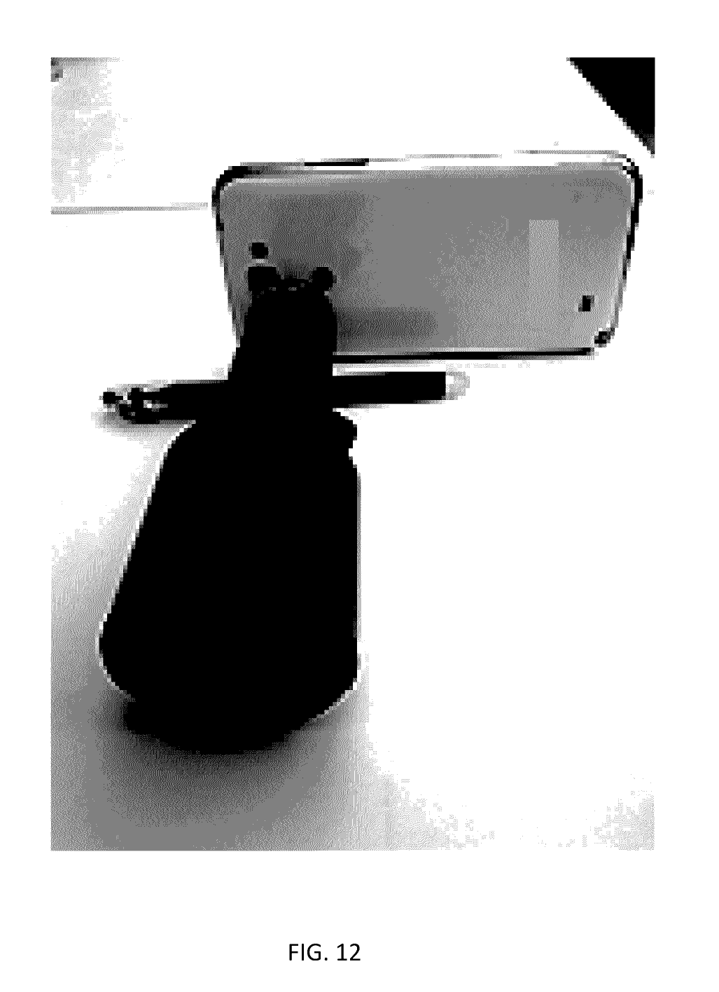

Referring to FIG. 12, the herein disclosed system can be incorporated into an instrument of modest design and cost by, for example, integrating and physically and communicatively coupling the herein described system with smartphone technologies. The smartphone technologies can include but are not limited to smartphones, tablets, laptops, and computers.

Those skilled in the art will recognize that numerous modifications can be made to the specific implementations described above. The implementations should not be limited to the particular limitations described. Other implementations may be possible. In addition, all references cited herein are indicative of the level of skill in the art and are hereby incorporated by reference in their entirety.

### REFERENCES

1.Barbin, D., ElMasry, G., Sun, D. W., & Allen, P. (2012). Near-infrared hyperspectral imaging for grading and classification of pork. Meat Science, 90, 259-268
2. Cluff, K., Naganathan, G. K., Subbiah, J., Samal, A., & Calkins, C. R. (2013). Optical scattering with hyperspectral imaging to classify longissimus dorsi mucle based on beef tenderness using multivariate modeling. Meat Science, 95, 42-50
3. ElMasry, G., Sun, D. W., & Allen, P. (2012) Near-infrared hyperspectral imaging for prediciting colour, pH and tenderness of fresh beef. Journal of Food Engineering, 110,127-140
4.Grobbel, J. P., Dikeman, M. E., Hunt, M. C., & Milliken, G. A. (2008). Effects of packaging atmosphere on beef instrumental tenderness, fresh color stability and internal cooked color. Journal of Animal Science, 86(5), 1191-1199.
5. Jackman, P., Sun, D. W., & Allen, P. (2011). Recent advanes in the use of computer vision technology in the quality assessment of fresh meats. Trends in Food Science & Technology, 22,185-197
6. Quevedo, R., Valencia, E., Cuevas, G., Ronceros, B., Pedreschi, F., & Bastias, J. M. (2013). Color changes in the surface of fresh cut meat: a fractal kinetic application. Food Research International, 54(2), 1430-1436
7. Rust, S. R., Price, D. M., Subbiah, J., Kranzler, G., Hilton, G. G., Vanoverbeke, D. L., & Morgan, J. B. (2008). Predicting beef tenderness using near-infrared spectroscopy. Journal of Animal Science, 86(1), 211-218
8. Wheeler, T. L., Shackelford, S. D., & Koohmaraie, M. (1997). Standardizing colletin and interpretation of WarnerBratzler shear force and sensor tenderness data. Proc. Recip. Meat Conf. 50:68-77
9. Wheeler, T. L., Shackelford, S. D., & Koohmaraie, M. (1999). Tenderness classification of beef: IV. effect of USDA quality grade on the palatability of “tender” beef longissimus when cooked well done. Journal of Animal Science, 77(4), 882-888.
10. Wyle, A. M., Vote, D. J., Roeber, D. L., Cannell, R. C., Belk, K. E., Scanga, J. A., Goldberg, M., Tatum, J. D., & Smith, G. C. (2003). Effectiveness of the SmartMV prototype BeefCam system to sort beef carcasses into expected palatability groups. Journal of Animal Science, 81(2), 441-448
11. Yacowitz, H., Davies, R. E., & Jones, M. L. (1978). Direct instrumental measurement /of skin color in broilers. Poultry Science, 57, 443-448
12. Xiong, Z., Sun, D. W., Zeng, X. A., & Xie, A. (2014). Recent development of hyperspectral imaging systems and their applications in detecting quality attributes of red meats: a review. Journal of Food Engineering, 132, 1-13
13. Z. Xu, A. K. Somani, and Y. L. Kim, “Scattering anisotropy-weighted mesoscopic imaging,” Journal of Biomedical Optics 19(9):090501, 2012.
14. Abidoye, Babatunde O., Harun Bulut, John D. Lawrence, Brian Mennecke, and Anthony M. Townsend. 2011. U.S. Consumers' Valuation of Quality Attributes in Beef Products. Journal of Agricultural and Applied Economics 43 (01):1-12.
15. AMSA. 1995. Research guidelines for cookery, sensory evaluation and instrumental tenderness measurements of fresh meat. Chicago, Ill.
16. Keeton, Jimmy T., Brian S. Hafley, Sarah M. Eddy, Cindy R. Moser, Bobbie J.

McManus, and Timothy P. Leffler. 2003. Rapid Determination of Moisture and Fat in Meats by Microwave and Nuclear Magnetic Resonance Analysis. Journal of AOAC International 86 (6):1193-1202.
17. Kim, Y.H.B., D. Setyabrata, T. Kim, and Y. L. Kim. 2016. Meat tenderness assessment using tissue anisotropy imaging analysis. Meat Science (112):153.
18. Kim, Yuan H. Brad, Genevieve Luc, and Katja Rosenvold. 2013. Pre rigor processing, ageing and freezing on tenderness and colour stability of lamb loins. Meat Science 95 (2):412-418.
19. Konger, Raymond L., Zhengbin Xu, Ravi P. Sahu, Badri M. Rashid, Shama R. Mehta, Deena R. Mohamed, Sonia C. DaSilva-Arnold, Joshua R. Bradish, Simon J. Warren, and Young L. Kim. 2013. Spatiotemporal Assessments of Dermal Hyperemia Enable Accurate Prediction of Experimental Cutaneous Carcinogenesis as well as Chemopreventive Activity. Cancer Research 73 (1):150-159.
20. Leroy, B., S. Lambotte, 0. Dotreppe, H. Lecocq, L. Istasse, and A. Clinquart. 2004. Prediction of technological and organoleptic properties of beef Longissimus thoracis from near-infrared reflectance and transmission spectra. Meat Science 66 (1):45-54.
21. Miller, M. F., L. C. Hoover, K. D. Cook, A. L. Guerra, K. L. Huffman, K. S. Tinney, C.

B. Ramsey, H. C. Brittin, and L. M. Huffman. 1995. Consumer Acceptability of Beef Steak Tenderness in the Home and Restaurant. Journal of Food Science 60 (5):963-965.
22. Moore, M. C., G. D. Gray, D. S. Hale, C. R. Kerth, D. B. Griffin, J. W. Savell, C. R. Raines, K. E. Belk, D. R. Woerner, J. D. Tatum, J. L. Igo, D. L. VanOverbeke, G. G. Mafi, T. E. Lawrence, R. J. Delmore, L. M. Christensen, S. D. Shackelford, D. A. King, T. L. Wheeler, L. R. Meadows, and M. E. O′Connor. 2012. National Beef Quality Audit-2011: In-plant survey of targeted carcass characteristics related to quality, quantity, value, and marketing of fed steers and heifers. Journal of Animal Science.
23. Morgan, J. B. 1995. Enhance taste-palatability. National Cattlemens Beef Association, Centennial, CO.
24. Polkinghorne, R. J., and J. M. Thompson. 2010. Meat standards and grading: A world view. Meat Science 86 (1):227-235.
25. Umberger, Wendy J., Peter C. Boxall, and R. Curt Lacy. 2009. Role of credence and health information in determining US consumers' willingness-to-pay for grass-finished beef. Australian Journal of Agricultural and Resource Economics 53 (4):603-623.
26. Visbal-Onufrak, M. A., R. L. Konger, and Y. L. Kim. 2016. Telecentric suppression of diffuse light in imaging of highly anisotropic scattering media. Optics Letters 41 (1):143-146.
27. Wu, J. J., T. R. Dutson, and Z. L. Carpenter. 1981. Effect of postmortem time and temperature on the release of lysosomal enzymes and their possible effect on bovine connective tissue components of muscle. Journal of Food Science 46 (4):1132-1135.
28. Xiong, Zhenjie, Da-Wen Sun, Xin-An Zeng, and Anguo Xie. 2014. Recent developments of hyperspectral imaging systems and their applications in detecting quality attributes of red meats: A review. Journal of Food Engineering 132:1-13.
29. Xu, Zhengbin, Ally-Khan Somani, and Young L. Kim. 2012. Scattering anisotropy-weighted mesoscopic imaging. Journal of Biomedical Optics 17 (9):0905011-0905013.

1. A hyperspectral imaging system, comprising:
a detector; and
a broadband light source.

2. The hyperspectral imaging system of claim 1, wherein the detector comprises a camera.

3. The hyperspectral imaging system of claim 2, wherein the camera is a charge-coupled device (CCD) camera.

4. The hyperspectral imaging system of claim 2, wherein the camera is a complementary metal oxide semiconductor (CMOS) camera.

5. The hyperspectral imaging system of claim 1, wherein the broadband light source comprises a white-light source.

6. The hyperspectral imaging system of claim 5, wherein the white-light source comprises light emitting diodes.

7. The hyperspectral imaging system of claim 5, wherein the white-light source comprises xenon.

8. The hyperspectral imaging system of claim 5, wherein the white-light source comprises tungsten lamps.

9. A method for assessing meat tenderness, comprising:
illuminating a tissue sample with a light;
varying at least one parameter of the light with a spectrometer; and
forming an image of the tissue sample by collecting backscattered light from the tissue using a lens system.

10. The method of claim 9, wherein the lens system comprises at least one of a small aperture 4-focal length lens system within an angular cone of 2°-5°, a telecentric lens, and an anti-scatter grid and a detector lens.

11. The method of claim 9, wherein the at least one parameter of the light comprises the wavelength of the light.

12. The method of claim 9, wherein the light is diffused light.

13. The method of claim 9, wherein the light is collimated light.

14. The method of claim 9, further comprising coupling and decoupling scattering and absorption contributions in the tissue sample.

15. A method for assessing meat tenderness, comprising:
illuminating broadband light from a light source onto a sample;
resolving wavelength information using a filter; and
obtaining an image set of different wavelengths of the sample using a detector.

16. The method of claim 15, wherein the broadband light comprises at least one of visible light and near infrared light.

17. The method of claim 16, wherein the visible and near-infrared light wavelength range is about 400 to about 1400 nm.

18. The method of claim 15, wherein the filter comprises a mechanical color filter.

19. The method of claim 15, wherein the filter comprises a liquid crystal tunable filter.

20. The method of claim 15, wherein the filter comprises an imaging spectrograph.
